(19) United States
(12) Patent Application Publication
(10) Pub. No.: US 2010/0260648 A1
(43) Pub. Date: Oct. 14, 2010

(54) Sterilization Device for a Dental Appliance

(76) Inventor: Chih-Ju LIN, Taishan Township (TW)

Correspondence Address: ARCHER & GREINER, P.C.;John F. Letchford, ONE CENTENNIAL SQUARE, P.O. Box 3000, HADDONFIELD, NJ, 08033, US

(21) Appl. No.: 12/140,869

(22) Filed: Jun. 17, 2008

(51) Int. Cl. A61L 2/20; A61L 2/10; A61L 2/00

(52) U.S. Cl. 422/186.12; 422/186.3; 422/187

(57) ABSTRACT

The present invention relates to a sterilization device for a dental appliance, which is primarily assembled from an upper cover, a holding body and a base, wherein a load supporting portion is formed at one end of the holding body, and the load supporting portion is fitted with a bacteria removal member. Moreover, a power supply device is disposed between the holding body and the base. Accordingly, an object to be sterilized is placed in the load supporting portion, and an electrical connection is effected with the bacteria removal member by means of the power supply device, thereby causing the bacteria removal member to release ozone (O₃) or ultra violet rays to carry out sterilization of the object to be sterilized and achieve bacteria removal effectiveness.

### BACKGROUND OF THE INVENTION

(a) Field of the Invention

The present invention provides a sterilization device, and more particularly provides a sterilization device that uses ozone or ultra violet rays to carry out sterilization of an object to be sterilized and achieve the effectiveness of removing bacteria.

(b) Description of the Prior Art

The oral cavity of a person is a critical organ for chewing food, and cleaning of the oral cavity must be carried out every time a meal is eaten and before and after sleeping to ensure health of the oral cavity and prevent halitosis, as well as improving interpersonal relationships and avoiding having to wear a dental appliance or false teeth because of oral disease. Apart from the aforementioned, many athletes (such as: boxers, wrestlers, rugby players, and so on) are subject to relatively severe contact or collision in their sports, which results in the oral cavity easily becoming injured, thus, such athletes also wear related dental appliances to protect their oral cavity and ensure self-protection. However, temperature and moisture content resulting from saliva secreted by the oral cavity gives rise to a situation conducive to multiplication of bacteria. Hence, when the user wears false teeth or a dental appliance, saliva within the oral cavity will also adhere to the false teeth or dental appliance, and if the user does not immediately clean the false teeth or dental appliance after removal, then the bacteria in the saliva will stay attached to the false teeth or dental appliance, which, over a long period of time, will conceal grime. Accordingly, the next time the user wears the false teeth or dental appliance, foreign bacteria is easily deposited within the oral cavity, and because the oral cavity itself only has resistance against the bacteria existing in saliva, thus, when foreign bacteria invades the oral cavity, then inflammation of the gums occurs or causes teeth decay, resulting in the user having to undergo long term treatment, causing unbearable distress.

In light of the aforementioned circumstances, pharmaceutical agents are promoted on the market for cleaning and sterilizing false teeth or dental appliances, as depicted in FIG. 1, wherein a container 1 is provided with a retaining space 10, and the container 1 is filled with a pharmaceutical agent 2 able to clean an object to be sterilized 3 (such as: false teeth or a dental appliance), and, after a period of time, the false teeth or dental appliance is removed from the container. Using such a method enables achieving the effectiveness to clean and sterilize the false teeth or dental appliance.

However, the following problems and shortcomings are still in need of improvement when using the aforementioned pharmaceutical agent for sterilizing a dental appliance:

1. The user must additionally purchase the pharmaceutical agent 2, and sterilize the false teeth or dental appliance at fixed times to prevent bacteria from multiplying.

2. Because the pharmaceutical agents 2 are all corrosiveness, thus, should the user forget to remove the false teeth or dental appliance from the pharmaceutical agent 2 within the scheduled time, or after a long period of using the pharmaceutical agent 2 to clean and sterilize the false teeth or dental appliance, then premature damage to the false teeth or dental appliance easily occurs, correspondingly, optimum protection of the oral cavity is not achieved, but has an exactly opposite effect.

3. If the user, after removing the false teeth or dental appliance from the pharmaceutical agent 2, and does not clean off the pharmaceutical agent 2 remaining on the surface, then, over a long period of time, secondary injury to the gums or teeth results.

4. When the user goes on a trip, then he must carry the containers filled with pharmaceutical agent 2, causing inconvenience while traveling.

Hence, it is the strong desire of the inventor and manufacturers engaged in related art and purpose of the present invention to resolve and surmount existent technical difficulties to resolve the problems and shortcomings of the aforementioned prior art.

### SUMMARY OF THE INVENTION

Hence, in light of the shortcomings of the aforementioned prior art, the inventor of the present invention, having collected related data, and through evaluation and consideration from many aspects, as well as having accumulated years of experience in related arts, through continuous testing and improvements has designed a new sterilization device for a dental appliance that uses ozone or ultra violet rays to carry out sterilization of an object to be sterilized and achieve the effectiveness of removing bacteria.

A primary objective of the present invention lies in providing a sterilization device for a dental appliance which is primarily assembled from an upper cover, a holding body and a base, wherein a load supporting portion is formed at one end of the holding body, and the load supporting portion is fitted with a bacteria removal member. The upper cover is pin connected to the load supporting portion, in addition, a plurality of clamping pegs extend away from the load supporting portion at the rear of the holding body. Furthermore, a retaining space is defined at one end of the base, and a plurality of clamping portions corresponding to the clamping pegs extend from the retaining space, and a retaining area is further formed at the rear of the base away from one end of the retaining space; moreover, a power supply device is disposed between the holding body and the base. Accordingly, an object to be sterilized is placed in the load supporting portion, and an electrical connection is effected with the bacteria removal member by means of the power supply device, thereby causing the bacteria removal member to release ozone (O₃) or ultra violet rays to carry out sterilization of the object to be sterilized and achieve bacteria removal effectiveness.

Another objective of the present invention lies in the user avoiding the extra expense of purchasing excessive cleaning products while preserving the false teeth or dental appliance and preventing bacteria from multiplying thereon, thereby saving a considerable amount of money over a period of time.

Yet another objective of the present invention lies in: because the sterilization device uses ozone or ultra violet rays to clean and sterilize false teeth or a dental appliance, thus, not only is the original structure of the false teeth or dental appliance preserved, preventing erosion thereof, moreover, the user avoids having to worry about pharmaceutical agents remaining on the false teeth or dental appliance and causing secondary injury to the gums or teeth.

Yet another objective of the present invention lies in: when the user goes on a trip, he need only carry the sterilization device, and when in use, there is no need to add any pharmaceutical agent; the user needs only to place the false teeth or dental appliance into the sterilization device, and ozone or ultra violet rays are used to carry out cleaning and sterilization, thereby providing easy portability and considerable convenience to the user.

To enable a further understanding of said objectives and the technological methods of the invention herein, a brief description of the drawings is provided below followed by a detailed description of the preferred embodiments.

### BRIEF DESCRIPTION OF THE DRAWINGS

FIG. 1 depicts the prior art using a pharmaceutical agent to sterilize.

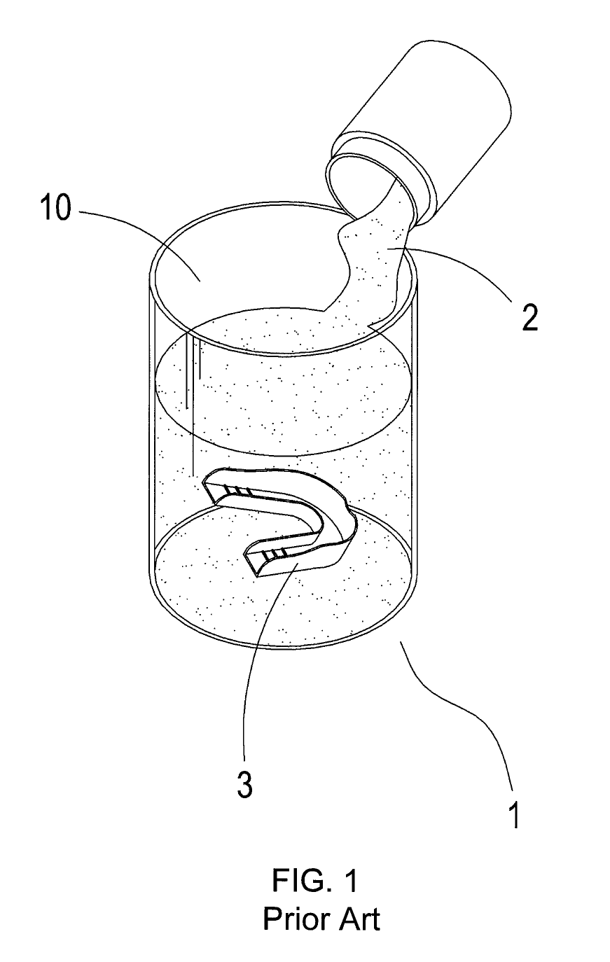

FIG. 2 shows an assembled elevational view of a preferred embodiment of the present invention.

FIG. 3 shows an exploded elevational view of the preferred embodiment of the present invention.

FIG. 4 shows a first operational schematic view of the preferred embodiment of the present invention.

FIG. 5 shows a second operational schematic view of the preferred embodiment of the present invention.

FIG. 6 shows a third operational schematic view of the preferred embodiment of the present invention.

FIG. 7 shows an operational schematic view of another preferred embodiment of the present invention.

FIG. 8 shows a schematic view depicting an operating state of the another preferred embodiment of the present invention.

### DETAILED DESCRIPTION OF THE PREFERRED EMBODIMENTS

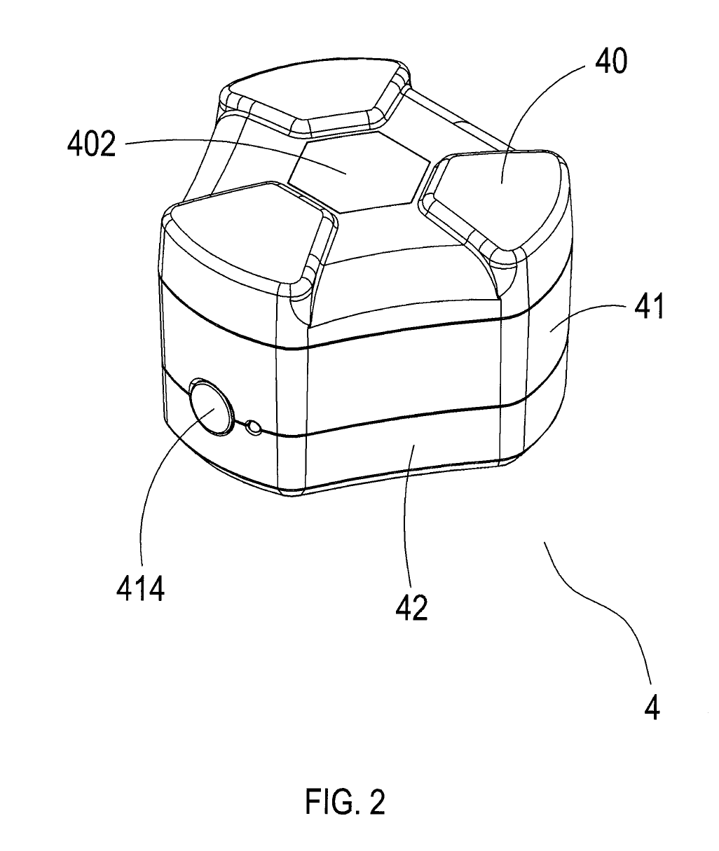

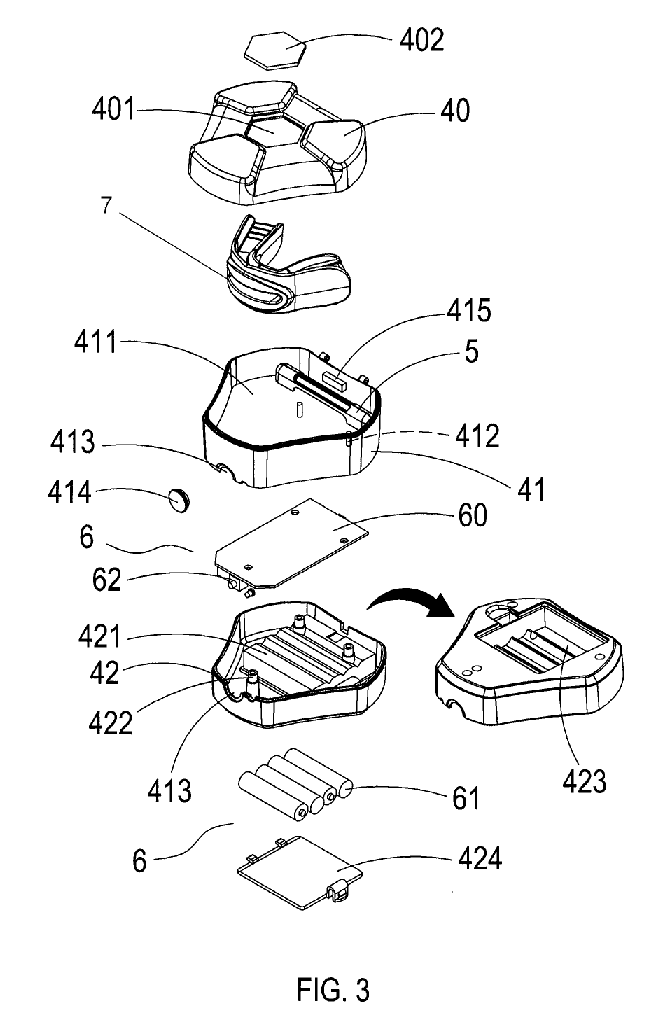

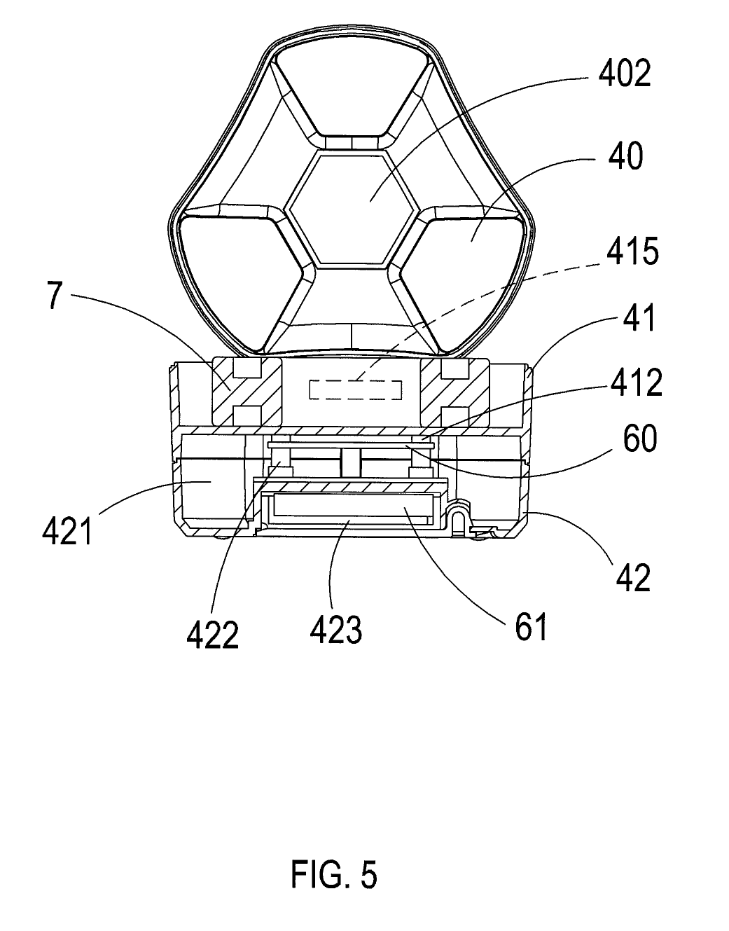

Referring to FIG. 2, FIG. 3 and FIG. 5, which show an assembled elevational view, an exploded elevational view and a second operational schematic view respectively of a preferred embodiment of the present invention, and it can be clearly seen from the drawings that the present invention is a sterilization device for a dental appliance, wherein a sterilization device 4 is primarily assembled from an upper cover 40, a holding body 41 and a base 42, wherein a load supporting portion 411 is formed at one end of the holding body 41, and the load supporting portion 411 is fitted with a bacteria removal member 5 (such as: a member able to emit ozone (O₃) or ultra violet rays). The upper cover 40 is pin connected to the load supporting portion 411, moreover, a fixed orifice 401, in which a transparent piece 402 is disposed, is formed at a preset position of the upper cover 40. In addition, a plurality of clamping pegs 412 extend away from the load supporting portion 411 at the rear of the holding body 41. Furthermore, a retaining space 421 is defined at one end of the base 42, and a plurality of clamping portions 422 corresponding to the clamping pegs 412 extend from the retaining space 421. After assembling the holding body 41 to the base 42 a hole 413 is formed at the inter-connecting area to correspond with a power control device 62, and a press member 414 is additionally located in the area of the hole 413. Furthermore, a retaining area 423 is formed at the rear of the base 42 away from one end of the retaining space 421, and the retaining area 423 is covered by fastening a protecting cover 424 thereon, thereby preventing power supplies 61 within the retaining area 423 from dropping out. A power supply device 6 is disposed between the holding body 41 and the base 42, and the power supply device 6 is primarily assembled from a circuit board 60, more than one of the power supplies 61 (such as: alkaline batteries, storage batteries or an externally connected power supply unit) and the power control device 62. Wherein the circuit board 60 is a power saving circuit board and is located at a preset position between the holding body 41 and the base 42, and forms an electrical connection with the bacteria removal member 5. Moreover, the power supplies 61 are installed in the retaining area 423 formed in the base 42, and the retaining area 423 is further covered with the protecting cover 424. The power supplies 61 are connected to the circuit board 60 to form an electrical connection therewith. Furthermore, the power control device 62 is located at a preset position on the circuit board 60, and is connected to the circuit board 60 to from an electrical connection therewith, thereby control opening and closing of the circuit board 60.

Because, a sensor 415 is located at a present position of an inner side of the holding body 41, thus, when the sterilization device 4 is activated, then the sensor 415 is simultaneously activated. Hence, when the user is using the sterilization device 4 to sterilize an object to be sterilized and accidentally opens the sterilization device 4, then the sensor 415 is simultaneously activated and immediately cuts the power to the sterilization device 4 by means of the circuit board 60, thereby preventing the bacteria removal member 5 from harming the eyes of the user.

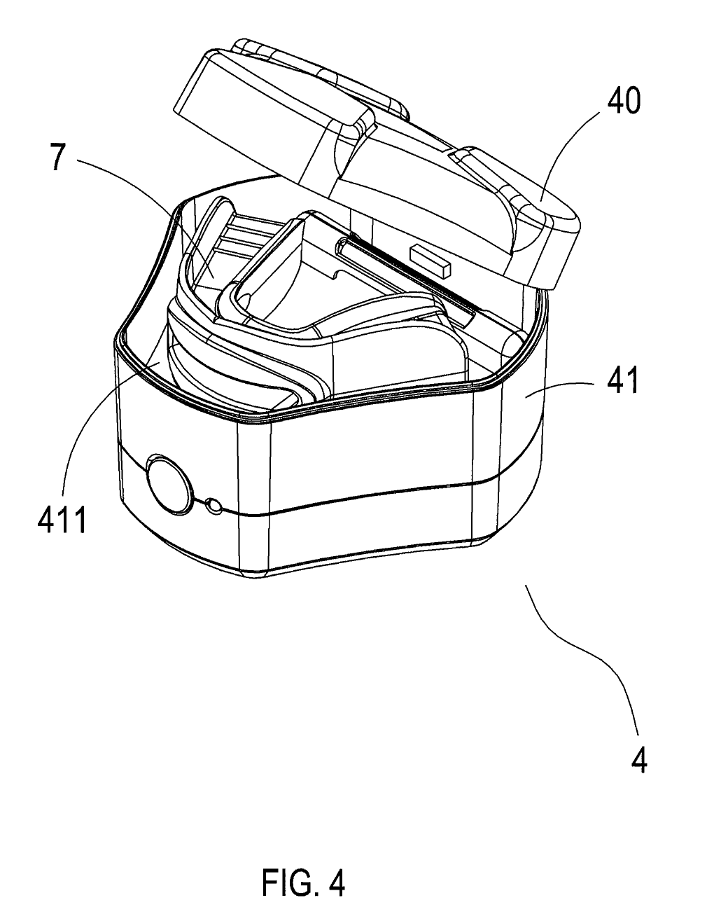

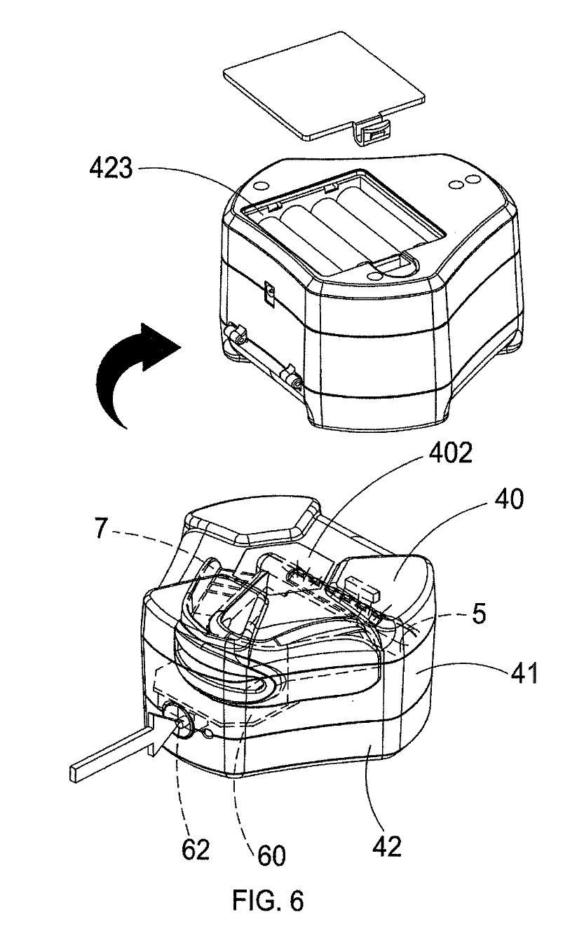

According to the aforementioned structure and constructional design, circumstances during operational use of the present invention are described hereinafter. Referring together to FIG. 4, FIG. 5 and FIG. 6, which show first, second and third operational schematic views respectively of a preferred embodiment according to the present invention, it can be clearly seen from the drawings that when the user wants to clean or sterilize an object to be sterilized 7 (such as: false teeth or a dental appliance), then the object to be sterilized 7 is placed in the load supporting portion 411, after which pressure is downwardly exerted on the upper cover 40 to fasten it onto the holding body 41. Accordingly, when the user goes on a trip, he need only carry the sterilization device 4 to sterilize the object to be sterilized 7, thereby providing easy portability and considerable convenience to the user.

Accordingly, the upper cover 40 is fastened to the holding body 41, the circuit board 60 is located at a preset position in the retaining space 421 defined in one end of the base 42, and the power supplies 61 are located at preset positions of the retaining area 423; furthermore, the holding body 41 and the base 42 are clamped together by means of the clamping pegs 412 and the respective clamping portions 422; thereby providing convenience of use. Taking common alkaline batteries as examples for the power supplies 61, after the user presses the power control device 62, then the alkaline batteries produce a predetermined electric current to activate the circuit board 60 and the bacteria removal member 5, whereupon the bacteria removal member 5 produces ozone (O₃) or ultra violet rays for sterilizing or cleaning the object to be sterilized 7. Moreover, the user can learn of the sterilization state of the false teeth or dental appliance through the transparent piece 402 located in the upper cover 40. Hence, because the sterilization device 4 uses using ozone or ultra violet rays to clean and sterilize false teeth or dental appliance, thus, the original structure of the false teeth or dental appliance is preserved during cleaning, and avoids damage from chemical substances.

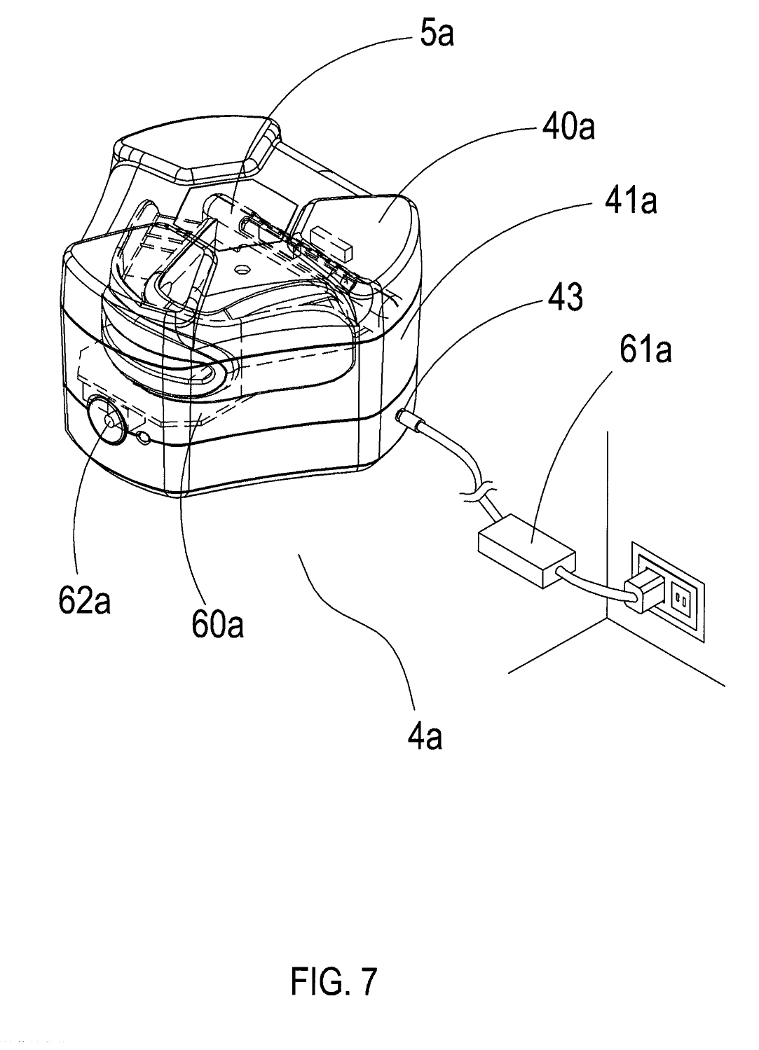

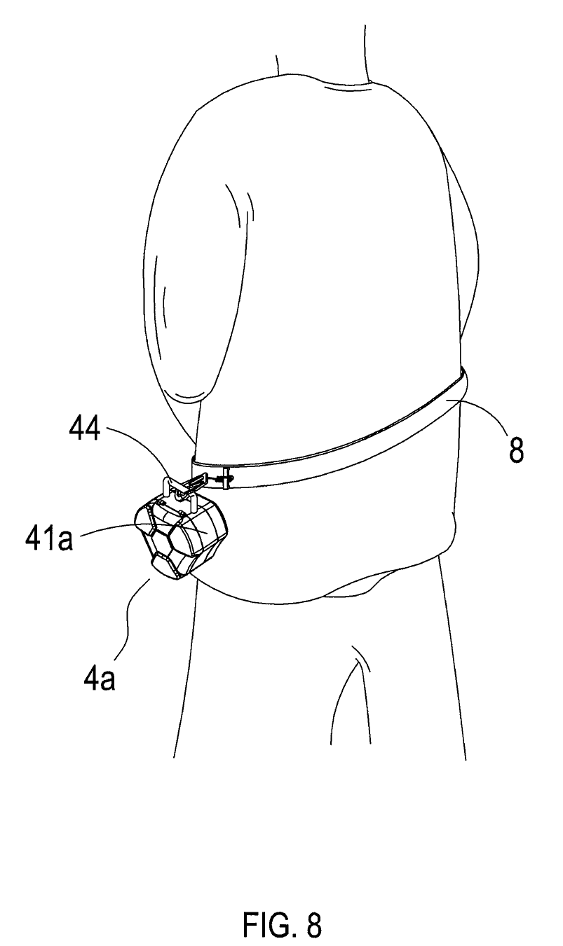

Referring to FIG. 7 and FIG. 8, which show an operational schematic view and a schematic view depicting operating state respectively of another preferred embodiment according to the present invention, and it can be clearly seen from the drawings that an upper cover 40a is fastened to a holding body 41a, and taking a general externally connected power supply unit (such as: a transformer) as an example of a power supply 61a, additionally, the externally connected power supply unit is either an alternating-current transformer or a direct current transformer, because a power jack 43, which enables a transformer to be plugged therein, is defined in a side of the holding body 41a, thus, the user must first insert the plug of the transformer into the power jack 43 before using a sterilization device 4a, after which the user presses a power control device 62a, whereupon the predetermined electric current produced by the transformer activates a circuit board 60a and a bacteria removal member 5a, which produces ozone (O₃) or ultra violet rays to sterilize and clean an object to be sterilized 7a. Hence, because the sterilization device 4a uses ozone or ultra violet rays to clean and sterilize false teeth or dental appliance, thus, the original structure of the false teeth or dental appliance is preserved during cleaning, and avoids damage from chemical substances.

Furthermore, because a fixing attachment 44, which enables clasping to a securing belt 8, is located at a preset position of an outer side of the holding body 41a, thus, when the user goes on a trip, the sterilization device 4a can be clasped to the waist or other related fitting using the securing belt 8, thereby preventing loss of the sterilization device 4a. Hence, referring to all the drawings, compared to the prior art, the following advantages exist when using the present invention:

1. The user avoids the extra expense of purchasing excessive cleaning products while preserving the false teeth or dental appliance and preventing bacteria from multiplying thereon, thereby saving a considerable amount of money over a period of time.

2. Because the sterilization device 4 uses ozone or ultra violet rays to clean and sterilize false teeth or a dental appliance, thus, not only is the original structure of the false teeth or dental appliance preserved, preventing erosion thereof, moreover, the user avoids having to worry about pharmaceutical agents remaining on the false teeth or dental appliance and causing secondary injury to the gums or teeth.

3. When the user goes on a trip, he need only carry the sterilization device 4, and when in use, there is no need to add any pharmaceutical agent, the user needs only to place the false teeth or dental appliance into the sterilization device 4, and ozone or ultra violet rays are used to carry out cleaning and sterilization, thereby providing easy portability and considerable convenience to the user.

In conclusion, the sterilization device for a dental appliance of the present invention is clearly able to achieve effectiveness and objectives when in use, and is indeed a practical and exceptional invention that complies with the essential elements as required for a new patent application. Accordingly, a new patent application is proposed herein.

It is of course to be understood that the embodiments described herein are merely illustrative of the principles of the invention and that a wide variety of modifications thereto may be effected by persons skilled in the art without departing from the spirit and scope of the invention as set forth in the following claims.

What is claimed is:

1. A sterilization device for a dental appliance, comprising:
a holding body, a load supporting portion is formed at one end of the holding body, and the load supporting portion is fitted with a bacteria removal member, an upper cover is pin connected to an end of the load supporting portion, in addition, a plurality of clamping pegs extend away from the load supporting portion at the rear of the holding body;
a base, a retaining space is defined at one end of the base, and a plurality of clamping portions corresponding to the clamping pegs extend from the retaining space, in addition, a retaining area is formed at the rear of the base away from one end of the retaining space; and
a power supply device, the power device is disposed between the holding body and the base.

2. The sterilization device for a dental appliance according to claim 1, wherein the power supply device further comprises:
a circuit board, the circuit board is located at a preset position between the holding body and the base, and forms an electrical connection with the bacteria removal member;
more than one power supply, the power supplies are installed in the retaining area formed in the base, and are connected to the circuit board to form an electrical connection therewith; and
a power control device, the power control device is located at a preset position on the circuit board, and is connected to the circuit board to from an electrical connection therewith.

3. The sterilization device for a dental appliance according to claim 2, wherein the power supplies are either alkaline batteries, storage batteries or an externally connected power supply unit.

4. The sterilization device for a dental appliance according to claim 3, wherein the externally connected power supply unit is either an alternating-current transformer or a direct current transformer.

5. The sterilization device for a dental appliance according to claim 2, wherein the power control device is a power switch.

6. The sterilization device for a dental appliance according to claim 2, wherein the circuit board is a power saving circuit board.

7. The sterilization device for a dental appliance according to claim 1, wherein a fixing attachment, which enables clasping to a securing belt, is located at a preset position of the holding body.

8. The sterilization device for a dental appliance according to claim 1, wherein a hole is formed in the peripheral wall of the holding body to correspond with the power control device.

9. The sterilization device for a dental appliance according to claim 1, wherein a fixed orifice, in which a transparent piece is disposed, is formed at a preset position of the upper cover.

10. The sterilization device for a dental appliance according to claim 1, wherein the bacteria removal member is either an ozone bacteria removal member or an ultra violet ray bacteria removal member.

11. The sterilization device for a dental appliance according to claim 1, wherein a sensor is located at a present position of an inner side of the holding body.
